(19) United States
(12) Patent Application Publication
(10) Pub. No.: US 2022/0079685 A1
(43) Pub. Date: Mar. 17, 2022

(54) SURGICAL NAVIGATION SYSTEM, MEDICAL IMAGING SYSTEM WITH SURGICAL NAVIGATION FUNCTION, AND REGISTRATION METHOD OF MEDICAL IMAGES FOR SURGICAL NAVIGATION

(71) Applicant: Hitachi, Ltd., Tokyo (JP)

(72) Inventors: Takafumi SHIMAMOTO, Tokyo (JP); Nobutaka ABE, Tokyo (JP)

(21) Appl. No.: 17/205,718

(22) Filed: Mar. 18, 2021

(30) Foreign Application Priority Data
Sep. 11, 2020 (JP) 2020-153259

(51) Int. Cl. A61B 34/20; A61B 90/00

(52) U.S. Cl. CPC A61B 34/20; A61B 90/361; A61B 2034/2065; A61B 2090/364; A61B 2034/2068; A61B 2090/363

(57) ABSTRACT

Two-step registration is performed so that a surface shape of a patient in real space accurately matches a surface shape in a medical image. The medical image of the patient is received from an imaging device, and position information is acquired for three or more regions on the surface of the patient and one representative position is set for each of the regions. Initial registration is performed using the information of the representative positions to establish association between the orientation of the patient in real space with the orientation of the patient image in the medical image. Then, detailed registration is performed to further establish the association, so that the surface shape of the patient represented by the positions of the plurality of points in the three or more regions matches the surface shape of the patient image in the medical image.

### CROSS-REFERENCE TO RELATED APPLICATION

The present application claims priority from Japanese application JP2020-153259, filed on Sep. 11, 2020, the contents of which is hereby incorporated by reference into this application.

### BACKGROUND OF THE INVENTION

### Field of the Invention

The present invention relates to a technique for performing registration between a patient position in real space and a medical image.

### Description of the Related Art

A surgical navigation system displays on a medical image, a positional relationship between a patient's position and a surgical instrument during a surgical operation, to provide information for assisting treatment or the surgical operation.

In order to perform surgical navigation, registration is required between the patient's position in real space and the position of the patient's image in the medical image. As one of the registration methods, an imaging marker is affixed to the patient to be imaged, and the position of the marker in real space is matched with the position of the marker on the medical image. This method may cause problems such as an increased burden on a medical worker due to extra work for affixing the marker and an increased burden on the patient to keep the marker be affixed from the time of the imaging until the surgical operation is performed, and displacement of the marker may hamper the registration.

In JP-A-2007-209531 (hereinafter, referred to as Patent Document 1), there is disclosed another registration method (surface registration) where surface information of a patient obtained by using a laser or the like, is associated by pattern matching, with the surface information of a three-dimensional image obtained from a medical image.

Further, in “Application of Surgical Simulation and Navigation System with 3D Imaging”, Kenshi KANEKO, et al., MEDICAL IMAGING TECHNOLOGY, Vol. 18, No. 2, March 2000, pp. 121-126 (hereinafter, referred to as Non-Patent Document 1), there is disclosed a method of performing point registration and surface registration in combination. This method first uses a marker or an anatomical landmark affixed to the patient to establish an association between the position of the marker or the anatomical landmark in real space, and the position of the marker or the anatomical landmark on the medical image. Thereafter, the surface registration is performed. This allows accurate registration of the surface shape of the patient in real space with the medical image.

In the surface registration described in Patent Document 1, if the angle (initial angle) formed between an orientation of the surface shape of the patient in real space and the orientation of the surface shape in the medical image is too large before performing the registration, the registration process by pattern matching may fall into a local solution, and in some cases, an accurate registration result cannot be obtained. For example, as shown in FIG. 12A-1, there is assumed the case where the pattern matching is performed while the orientation of the surface shape 902 of the patient in real space and the orientation of the surface shape of the patient 901 in the medical image are greatly different. In the state where the angle between the surface shape of the patient 902 in real space and the surface shape of the patient 901 in the medical image is deviated, if there is an area with which the curved surface occasionally coincides, as shown in FIG. 12A-2, the registration may be performed with keeping the angle being displaced.

On the other hand, as described in Non-Patent Document 1, prior to the surface registration, the point registration is performed to allow the orientation of the surface shape of the patient 902 in real space to match the orientation of the surface shape of the patient 901 in the medical image (FIG. 12B-1), followed by the surface registration, and this enables accurate registration (FIG. 12B-2).

However, when the point registration is performed before the surface registration, it is necessary to measure the position of the marker or the anatomical landmark affixed to the patient in real space as described in Non-Patent Document 1. Therefore, for example, in the case of a head, a user is required to point at a position of the forehead, right and left temporal regions, or another portion of the patient, with a pointer or a similar tool, to measure the position. Thereafter, in order to obtain body surface data of the patient used for the surface registration, it is necessary to scan the head surface of the patient by a laser or the like.

Thus, when both the point registration and the surface registration are performed, the user is required to perform two steps of operations: obtaining a point position for the point registration, and acquiring a surface shape of the patient for the surface registration. With the complexity of these operations, there arise problems such as increase of burden on the user, as well as increase of the operation time.

An object of the present invention is to perform a two-step registration so that the surface shape of the patient in real space can accurately match the surface shape on the medical image, also allowing reduction of the burden on the user.

### SUMMARY OF THE INVENTION

To achieve the object above, a surgical navigation system includes a storage unit configured to receive from an external device, a medical image being imaged for a patient and to store the medical image, a position detection sensor configured to detect position information of a point on a surface of the patient in real space, and an registration unit configured to establish association between a position of the patient in real space and a position of a patient image in the medical image. The registration unit acquires from the position detection sensor, the position information of a plurality of points in three or more regions on the surface of the patient in real space, sets one representative position for each of the three or more regions, then using information of the representative position for each of the regions, performs initial registration to establish association between an orientation of the patient in real space and an orientation of the patient image in the medical image, and thereafter, performs detailed registration to establish association between the position of the patient in real space and the position of the patient image in the medical image, so that the surface shape of the patient represented by the positions of the plurality of points within the three or more regions matches the surface shape of the patient image in the medical image.

According to the present invention, by setting the representative position for each of the three or more regions, both the initial registration using the representative positions and the registration using the surface shape represented by the plurality of points in the regions can be performed to accurately match the surface shape of the patient in real space with the surface shape in the medical image. Moreover, acquisition of the position information of the plurality of points in the regions, through the operation by the operator, is required to be performed only one time, and thus this allows reduction of burden on the user.

### BRIEF DESCRIPTION OF THE DRAWINGS

FIG. 1 is a block diagram showing a configuration of a surgical navigation system according to a first embodiment of the present invention;

FIG. 2 is a flowchart of a process of a registration unit of the surgical navigation system according to the first embodiment;

FIG. 3 illustrates point-group acquisition regions 311, 312, and 313, and surface point groups 321, 322, and 323, together with their centers of gravity 331, 332, and 333, in a patient in real space of the first embodiment;

FIG. 4 is a flowchart of a detailed process of step S201 of FIG. 2;

FIG. 5 illustrates a screen example in which the registration unit 21 of the first embodiment displays on a display device 6, the point-group acquisition region 311 to be presented for an operator;

FIG. 6 illustrates the screen example in which the registration unit 21 of the first embodiment displays on the display device 6, the point-group acquisition region 312 to be presented for the operator;

FIG. 7 illustrates the screen example in which the registration unit 21 of the first embodiment displays on the display device 6, the point-group acquisition region 313 to be presented for the operator;

FIG. 8 is a flowchart of a detailed process of step S203 of FIG. 2;

FIG. 9 illustrates vectors calculated by the process of FIG. 8;

FIG. 10 illustrates an example of a patient posture entry screen 800 according to a second embodiment;

FIG. 11 is a flowchart of a process of a posture input unit and the registration unit 21 according to the second embodiment; and

FIGS. 12A-1 and 12A-2 illustrate an example of registration according to surface registration by pattern matching, and FIGS. 12B-1 and 12B-2 illustrate an example of the surface registration after the point registration is performed.

### DETAILED DESCRIPTION OF THE PREFERRED EMBODIMENTS

There will now be described preferred embodiments of a surgical navigation system according to the present invention with reference to the accompanying drawings. In the following description and accompanying drawings, the components having the same functional configuration will be described with the same reference numerals and redundant descriptions will not be provided.

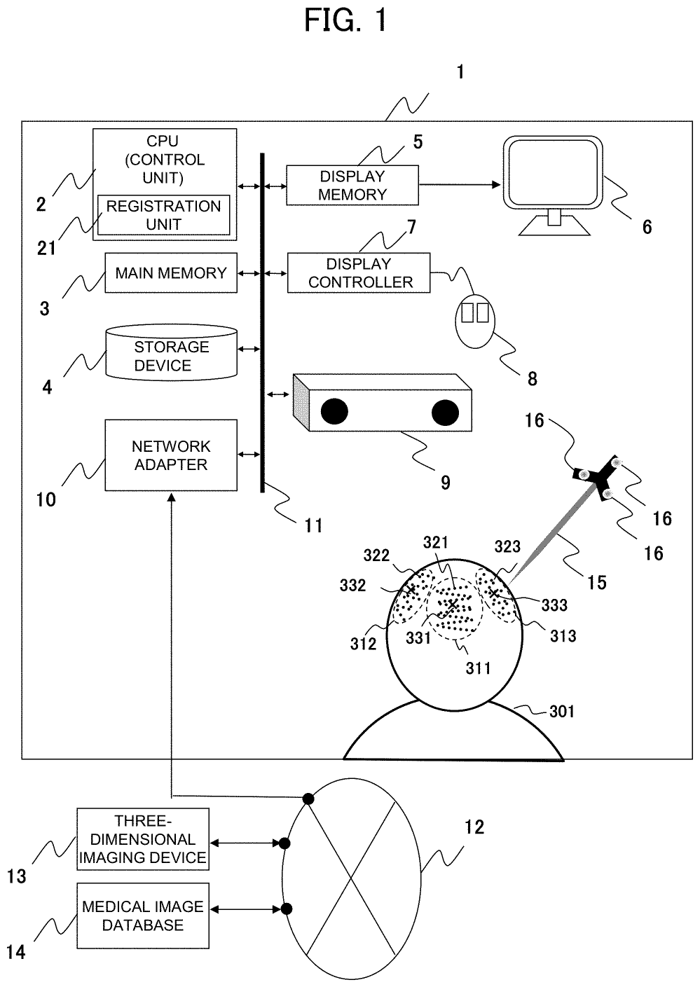

FIG. 1 is a diagram showing the configuration of the surgical navigation system 1. The surgical navigation system 1 includes a CPU (Central Processing Unit) 2, a main memory 3, a storage device 4, a display memory 5, a display device 6, a display controller 7 connected to a mouse 8, a position detection sensor 9 for detecting a position of a pointer 15, and a network adapter 10, and these elements are connected via a system bus 11 in such a manner as being capable of transmitting and receiving signals.

The surgical navigation system 1 is connected to a three-dimensional imaging device 13 and a medical image database 14 via a network 12, in such a manner as being capable of transmitting and receiving signals. Here, “capable of transmitting and receiving signals” refers to a state in which signals can be transmitted and received, mutually or from one side to the other, electrically or optically wired, or wirelessly.

The CPU 2 is a control unit configured to control the operation of each constitutional element, and to perform a predetermined computation. Hereafter, the CPU 2 will also be referred to as the control unit 2.

The main memory 3 is intended to hold programs and the progress of the computation executed by the CPU 2.

The storage device 4 is provided for storing medical image information captured by the three-dimensional imaging device 13 such as a CT device and an MRI device, and specifically, the storage device may be a hard disk, or the like. The storage device 4 may be configured to pass data with a portable recording medium, such as a flexible disk, an optical (magnetic) disk, a ZIP memory, and a USB memory. Medical image information is acquired from the three-dimensional imaging device 13 and the medical image database 14 via the network 12 such as a LAN (Local Area Network). Further, the storage device 4 stores a program to be executed by the CPU 2 and data required for executing the program.

The display memory 5 temporarily stores data to be displayed on the display device 6 such as a liquid crystal display and a CRT (Cathode Ray Tube). The mouse 8 is a manipulation device with which the operator provides an instruction for operating the surgical navigation system 1. The mouse 8 may be another pointing device, such as a trackpad and a trackball.

The display controller 7 detects the state of the mouse 8, acquires the position of the mouse pointer on the display device 6, and delivers information including the acquired position to the CPU 2.

The position detection sensor 9 is connected to the system bus 11 in such a manner as being capable of transmitting and receiving signals.

The network adapter 10 is provided for connecting the surgical navigation system 1 to the network 12 such as a LAN, telephone line, and the Internet.

The pointer 15 is a rod-shaped rigid body on which a plurality of reflecting spheres 16 can be mounted.

The position detection sensor 9 can recognize spatial coordinates of the reflecting spheres 16. Therefore, the position detection sensor 9 can detect the tip position of the pointer 15 on which a plurality of reflecting spheres 16 are mounted. Further, in a surgical operation, by using the surgical instrument on which more than one reflecting spheres 16 are mounted, it is possible to detect the tip position of the surgical instrument. The position information of the reflecting spheres 16 and the shape of the pointer 15, detected by the position detection sensor 9, are inputted in the CPU 2.

In the storage device 4, the program and data required for executing the program are stored in advance. The CPU 2 loads the program and data in the main memory 3, and executes the program, thereby serving as the control unit to implement various functions. Specifically, the CPU 2 uses the position information of the reflecting spheres 16 and the shape information of the pointer 15 or the surgical instrument, received from the position detection sensor 9, to perform computation according to a predetermined program, whereby the spatial position of the tip of the pointer 15 or the surgical instrument is calculated. Thus, the navigation device 1 can recognize the spatial position of the tip of the pointer 15 or the surgical instrument and can grasp the surface shape of the patient from the tip position information of the pointer 15. It is further possible to display the tip position of the surgical instrument on the medical image.

Further, the CPU 2 executes a registration program stored in advance in the storage device 4, thereby acquiring position information of the point groups in three or more regions on the surface of the patient in real space, and further functions as the registration unit 21 to perform the registration (registration) between the surface shape of the patient and the medical image. The registration process by the registration unit 21 will be described in detail according to the first and the second embodiments as the following.

It should be noted that it is also possible to implement by hardware, some or all of various functions such as a function of the CPU (control unit) 2 as a processing unit for calculating the tip position of the pointer 15 or the surgical instrument, and a function as the registration unit 21. For example, it is sufficient to perform circuit-designing, using a custom IC such as an ASIC (Application Specific Integrated Circuit) and a programmable IC such as FPGA (Field-Programmable Gate Array), to implement the function of the processing unit for calculating the tip position of the pointer 15 or the surgical instrument, the function of the registration unit 21, and others.

### First Embodiment

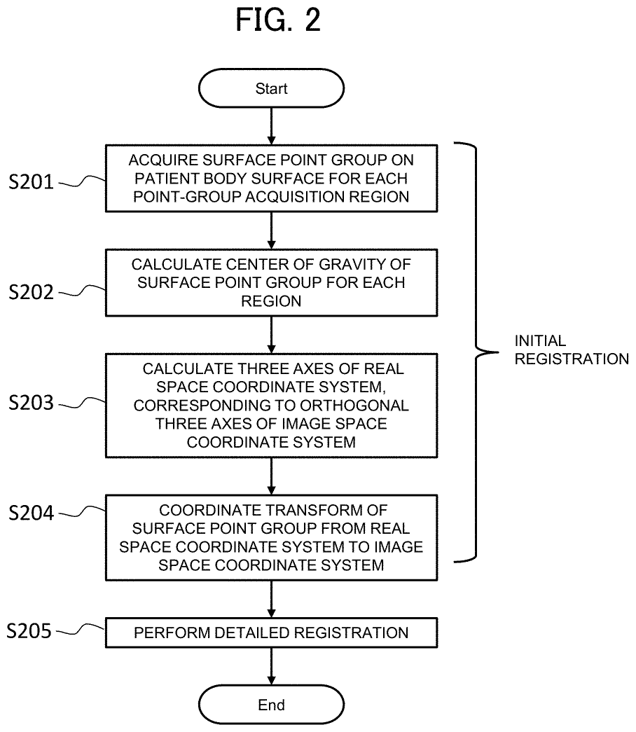

As the first embodiment, there will now be described in detail a process of the registration between the surface shape of the patient 301 and the medical image according to the navigation system as shown in FIG. 1. FIG. 2 is a flowchart of the registration process according to the surgical navigation system of the present invention.

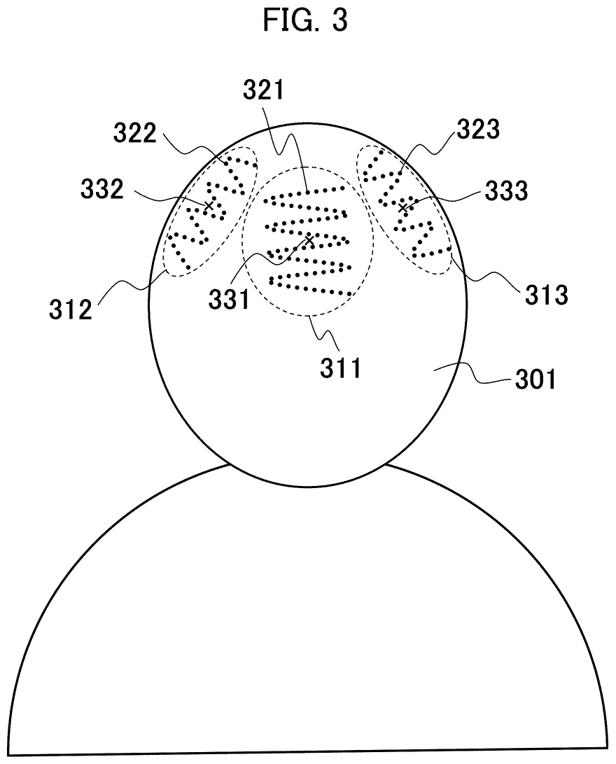

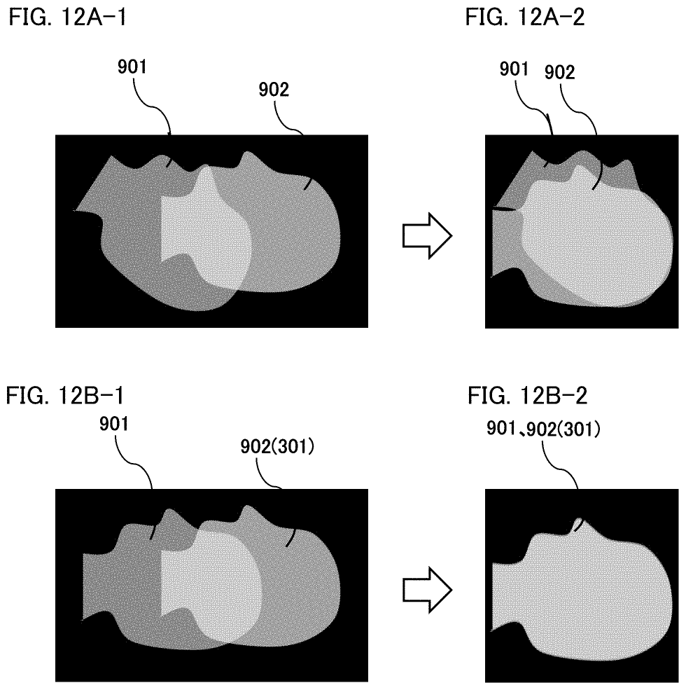

In the present embodiment, the registration unit 21 acquires from the position detection sensor 9, position information of a plurality of points in the regions 311, 312 and 313 which are three or more regions on the surface of the patient 301 in real space (see FIG. 3). The registration unit 21 sets representative positions 331, 332, and 333 respectively for the three or more regions 311, 312, and 313, and performs initial registration to establish association between the orientation of the patient 301 (902) in real space and the orientation of the patient 901 in the medical image as shown in FIG. 12B-1, using the information of the representative positions 331, 332, and 333 for the respective regions 311, 312, and 313. Thereafter, the registration unit 21 performs detailed registration to establish association between the patient position in real space with the patient position of the medical image, so that the surface shape of the patient 301 (902) represented by the positions of the plurality of points in the three or more regions 311, 312, and 313 match the surface shape of the image of the patient 901 in the medical image (see FIG. 12B-2).

The registration unit 21 is capable of calculating the representative positions 331, 332, and 333 respectively in the regions 311, 312, and 313, from the position information of the plurality of points in the regions. For example, the registration unit 21 calculates the center of gravity of the plurality of points as to which the position information has been obtained, for each of the regions 311, 312, and 313, and sets the centers of gravity as the representative positions 331, 332, and 333.

There will now be described the registration process of the surgical navigation system of the present invention. First, an outline of the registration process will be described with reference to the flowchart of FIG. 2. It should be noted that the medical image data used as an object of the patient registration is acquired from the three-dimensional imaging device 13 and the medical image database 14, and stored in the storage device 4.

### (Step S201)

As shown in FIG. 3, the registration unit 21 of the CPU (control unit) 2 acquires positions of a predetermined number (more than one) of points (hereinafter, referred to as “surface point groups”), respectively for the regions (hereinafter, referred to as “point-group acquisition regions”) 311, 312, and 313 provided on the patient surface in real space.

The point-group acquisition regions 311, 312, and 313 are three regions, out of the regions including two regions 312 and 313 facing in the left-right direction of the patient, and the region 311 and others facing in the front-rear direction of the patient 301. These three regions 311, 312, and 313 are preferably aligned along the circumferential direction of the patient 301.

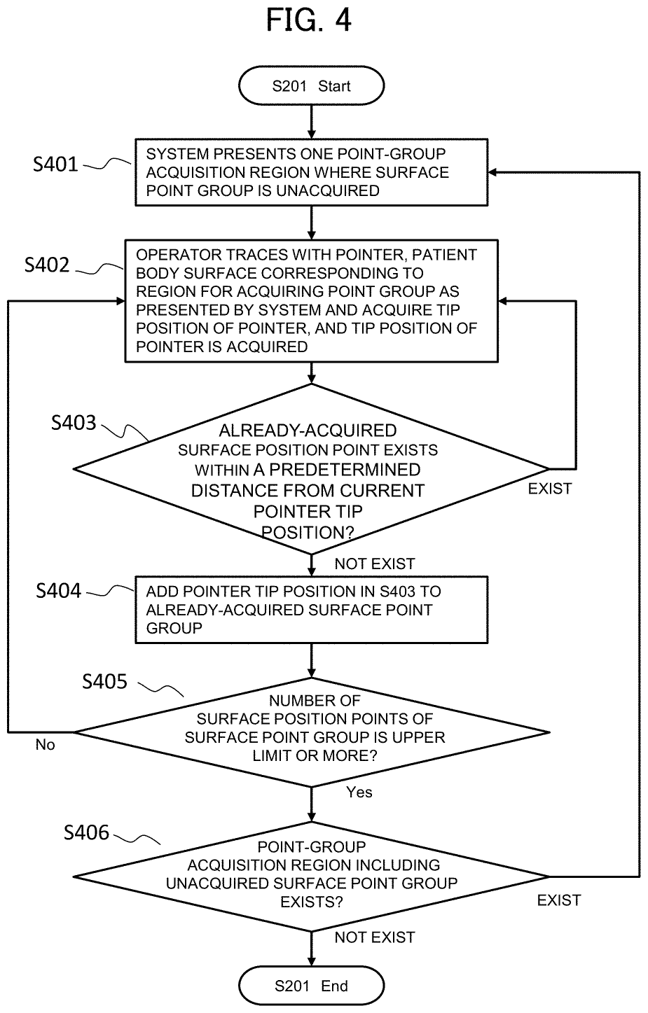

A method of acquiring the surface point groups 321, 322, and 323 will be described in detail later, with reference to the flowchart shown in FIG. 4.

### (Step S202)

Next, the registration unit 21 calculates representative positions 331, 332, and 333 respectively for the regions 311, 312, and 313. Here, the centers of gravity position G_{region} of the surface point groups 321, 322, and 323 are calculated according to Equation 1, respectively for the point-group acquisition regions 311, 312, and 313 acquired in step S201, and they are used as representative positions 331, 332, and 333.

$$\begin{matrix}Equation 1 & \\ {G}_{region} = \frac{1}{N} {\sum }_{k = 1}^{N} {P}_{region , k} & ( 1 )\end{matrix}$$

where “region” of G_{region} indicates any of the point-group acquisition regions 311, 312, and 313, N is the number of points constituting any of the surface point groups 321, 322, and 323, and P_{region,k} is a three-dimensional vector indicating the position of the k-th point in any of the surface point groups 321, 322, and 323 in any of the point-group acquisition regions 311, 312, and 313 represented by “region”.

### (Step S203)

The registration unit 21 uses the representative positions 331, 332, and 333 calculated in step S202 to obtain by computation the directions of the three axes of the real space coordinate system, corresponding to the orthogonal three axes of the image space coordinate system of the medical image.

For example, the registration unit 21 selects the facing regions 312 and 313, out of the three point-group acquisition regions 311, 312, and 313, and calculates a first vector connecting their representative positions 332 and 333. Further, a second vector orthogonal to a plane including the representative positions of the three point-group acquisition regions 311, 312, and 313 is calculated, and a third vector orthogonal to the first and second vectors is also calculated. Then, the first, the second, and the third vectors are respectively associated with the orthogonal three axes in image space of the medical image. The process of this step S203 will be described in detail later.

### (Step S204)

The registration unit 21 transforms the coordinates of the surface point groups 321, 322, and 323 of real space coordinate system, into the coordinates of the three-axis coordinate system, corresponding to the orthogonal three axes of the image space coordinate system that is obtained by step S203.

This completes the initial registration that associates between the orientation of the patient 301 in real space and the orientation of the patient image in the medical image.

### (Step S205)

Next, the registration unit 21 treats as one point group, the surface point groups 321, 322, and 323 which have been subjected to coordinate transformation in step S204, and performs detailed registration for establishing association between the position of the patient 301 in real space and the patient position in the medical image, so that the surface shape of the patient 301 represented by the point groups matches the surface shape obtained from the 3D image of the patient in the medical image. For example, a publicly known method such as an Iterative Closest Point method is used for this registration. Since the Iterative Closest Point method is a widely known method described in detail, in “A Method for Registration of 3-D Shapes”, Paul J. Besl and Neil D. McKay, IEEE TRANSACTIONS ON PATTERN ANALYSIS AND MACHINE INTELLIGENCE Vol. 14 No. 2, FEBRUARY 1992, pp. 239-255 (hereinafter, referred to as Non-Patent Document 2), a detailed description thereof will not be given here.

As described above, two-step registration can be performed according to the present embodiment. That is, in steps S201 to S204, the positions of the point groups 321, 322, and 323 in the three or more regions 311, 312, and 313 on the surface of the patient 301 are acquired, and using the representative positions 331, 332, and 333, the initial registration is performed between the orientation of the patient 301 and the orientation of the patient image of the medical image. Then, in step S205, the detailed registration is performed so that the surface shape of the patient 301 matches the surface shape obtained from the 3D image of the patient in the medical image. Therefore, it is possible to perform registration accurately between the patient 301 in real space and the patient image of the medical image.

Moreover, as will be described in detail thereafter, since the operator only needs to trace the regions 311, 312, and 313 with the pointer 15, burdens on both the operator and the patient 301 can be reduced, even though the registration is performed in two steps.

With reference to the flowchart shown in FIG. 4, there will be provided more detailed description of the process in step S201 as described above to acquire the positions of the point groups 321, 322, and 323 on the surface of the patient 301.

### (Step S401)

First, the registration unit 21 sequentially displays three or more regions 311, 312, and 313 on the display device 6 and prompts the operator to trace the surface of the patient 301 in the regions 311, 312, and 313 with the pointer 15.

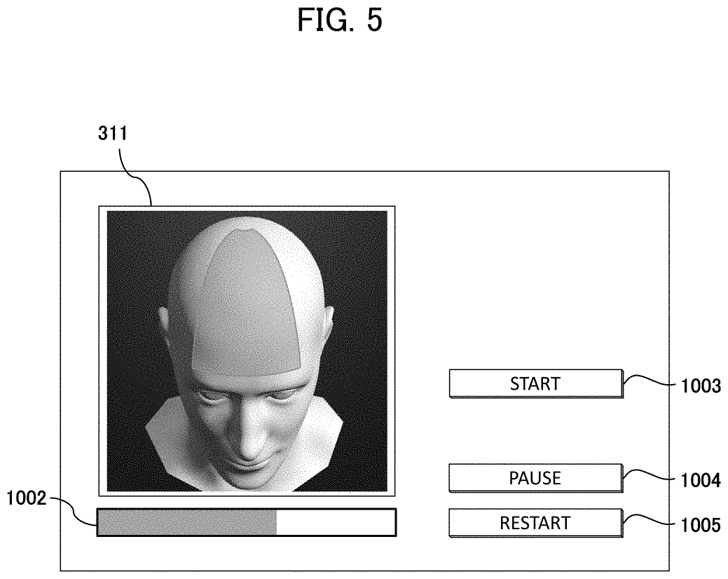

For example, as shown in FIGS. 5 to 7, the registration unit 21 displays on the display device 6 for the operator, as the region for acquiring the point group, one (e.g., region 311) of the point-group acquisition regions 311, 312, and 313 as to which the positions of the surface point group 321, 322, or 323 have not been acquired yet, and prompts the operator to trace the surface within the region 311 of the patient 301. In here, the point-group acquisition regions 311, 312, and 313 may correspond to three or more regions of the forehead, the right temporal region, the left temporal region, and the occipital region.

There has been described the case that the number of the point-group acquisition regions 311, 312, and 313 is three. However, the number of the point-group acquisition regions is not necessarily three, but there may be four or more regions. In that case, the fourth region and others may be used for correction.

### (Step S402)

The operator traces with the pointer 15, the body surface of the patient 301 within the region (region 311) displayed on the display device 6. For example, when the area 311 as the region to acquire the point group is displayed on the display device 6 as shown in FIG. 5, the operator selects the start button 1003, and then traces with the pointer 15 the region corresponding to the point-group acquisition region 311 on the patient surface.

The position detection sensor 9 detects the positions of the reflecting spheres 16 of the pointer 15 with which the operator traces the surface of the patient 301. The CPU 2 receives the positions of the reflecting spheres 16 and then performs a predetermined computation to calculate the tip position of the pointer 15. As a result, the registration unit 21 acquires the surface position of the patient 301.

### (Steps S403 and S404)

In order to have an interval of a predetermined distance or more, between the points in the surface point group 321, the registration unit 21 determines whether any already-acquired surface position point exists within a predetermined distance from the tip position of the pointer 15 calculated from the acquired positions (step S403). If there is not such surface position point, the process proceeds to step S404 to determine this position as the position information of the next point, and records the position in the main memory 3 (step S404). Thus, it is possible to acquire the point position at a predetermined distance or more away from the point position already acquired.

In step S403, if there exists any already-acquired surface position point within the predetermined distance from the tip position of the current pointer 15, the process returns to step S402 and the operator continues to trace the patient surface.

### (Step S405)

As a result of adding the point to the main memory 3 in step S404, the registration unit 21 determines whether or not the number of the acquired surface position points is equal to or more than the predetermined upper limit number. If the upper limit is not reached, the process returns to step S402.

At this time, the registration unit 21 displays on the display device, the progress bar 1002 representing the number of the points and the upper limit number thereof stored in the main memory 3. Thus, the progress bar 1002 shows the acquisition progress of the surface point group 321.

In the registration unit 21, when the number of the acquired surface position points reaches the upper limit number determined in advance, acquisition of the upper limit number of points of the surface point group 321 has been completed for the region 311, and then the process proceeds to step S406.

### (Step S406)

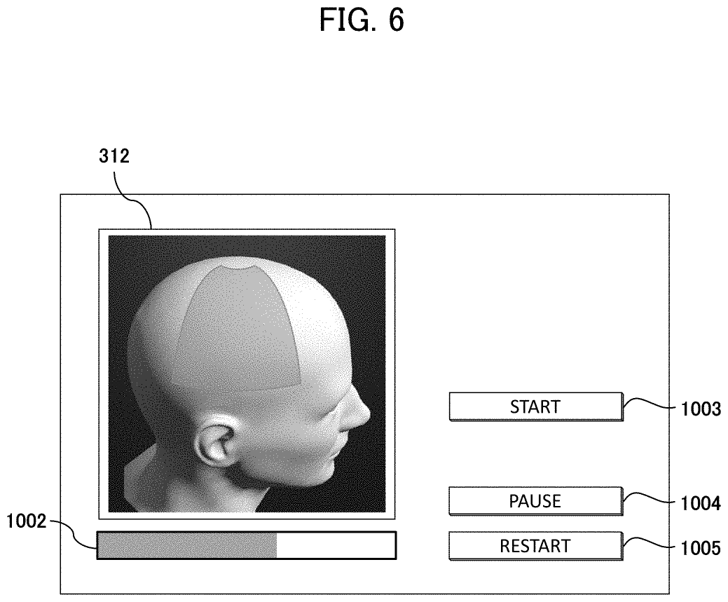

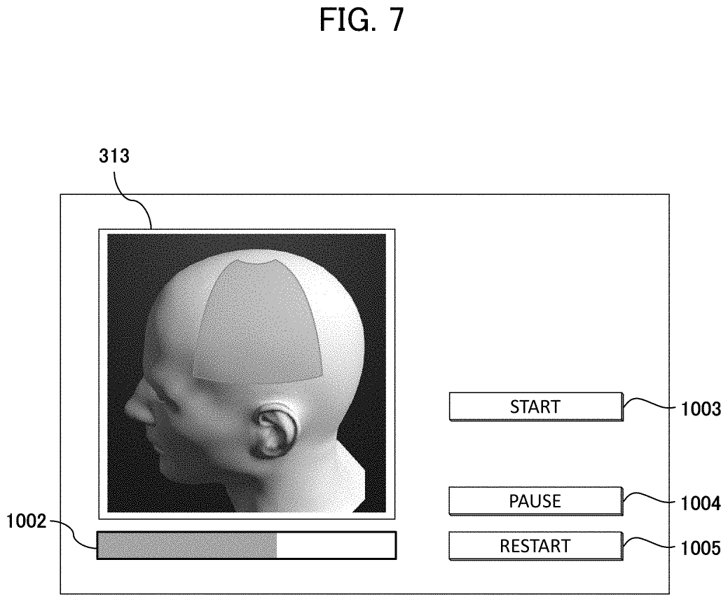

The registration unit 21 determines whether the point-group acquisition regions 312 and 313 exist, as to which the surface point group has not been acquired. If such point-group acquisition region is present, the process returns to step S401, and displays the point-group acquisition region 312 as in FIG. 6, and repeats the processes from steps S401 to S405. If the upper limit number of the points of the surface point group 322 is acquired for the point-group acquisition region 312, the process returns from step S406 to step S401, and the point-group acquisition region 313 is displayed as shown in FIG. 7, then steps S401 to S405 are repeated.

After the surface point groups of the upper limit number are acquired for all the point-group acquisition regions 311, 312, and 313, the step S201 ends and the process proceeds to step S202.

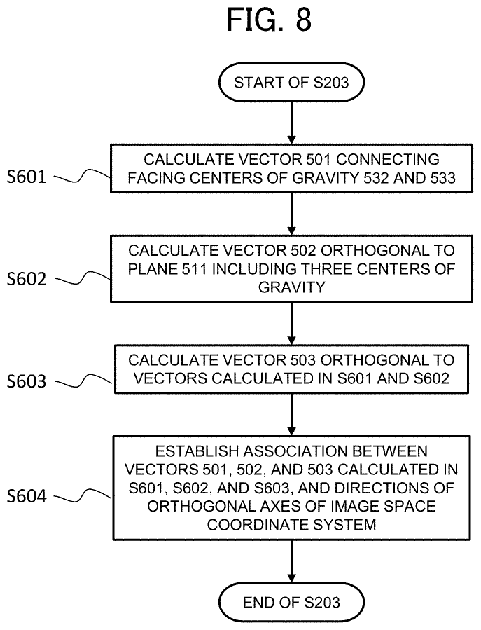

Hereinafter, with reference to FIG. 8, the process of the above-described step S203 will be described in more detail.

In steps S601 to S604 shown in FIG. 8, vectors in real space coordinate system corresponding to the orthogonal three axes of the image space coordinate system are calculated using the representative positions 331, 332, and 333.

### (Step S601)

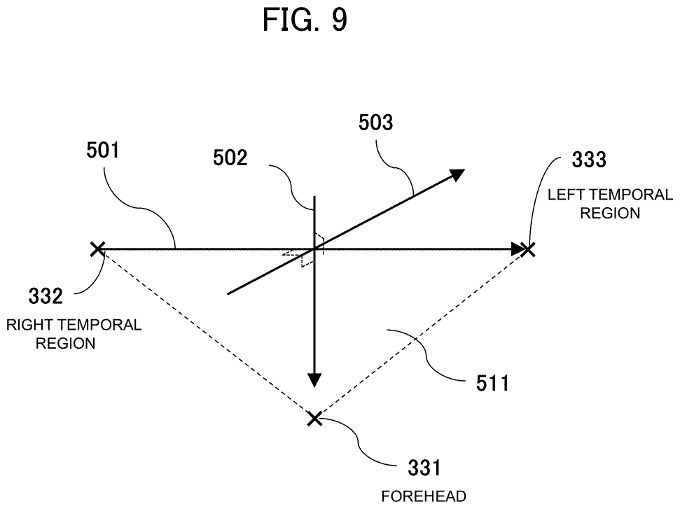

First, the registration unit 21 calculates a vector 501 connecting the positions of the centers of gravity 332 and 333 respectively of the two facing regions 312 and 313 of the patient, out of the positions of the centers of gravity (representative positions) 331, 332, and 333 respectively in the point-group acquisition regions 311, 312, and 313, as calculated in step S202 (see FIG. 9). Here, the vector 501 is calculated, connecting the center of gravity position 332 of the region 312 in the right temporal region, with the center of gravity position 333 of the region 313 in the left temporal region. Thus, the vector 501 in the left-right direction of the patient 301 can be calculated.

### (Step S602)

Next, the registration unit 21 obtains the plane 511 including the center of gravity positions (representative positions) 331, 332, and 333 of the three point-group acquisition regions 311, 312, and 313 calculated in step S202, and calculates a vector 502 orthogonal to the plane. Thus, the vector 502 in the body axis direction of the patient 301 can be calculated.

### (Step S603)

The registration unit 21 calculates a vector 503 orthogonal to both the vectors 501 and 502 that are calculated by steps S601 and S602. Thus, the vector 503 in the front-rear direction of the patient can be calculated.

### (Step S604)

Since the vectors 501 to 503 calculated in steps S601 to S603 are vectors in the left-right direction, in the body axis direction, and in the front-rear direction, they are respectively associated with the orthogonal three axes (the left-right direction, the body axis direction, and the front-rear direction) of the image space coordinate system previously included in the medical image data.

In FIG. 5, since the point-group acquisition regions 311, 312, and 313 correspond to the forehead, the right temporal region, and the left temporal region, the vectors 501 to 503 are vectors in the left-right direction, the body axis direction, and the front-rear direction, respectively. However, when the point-group acquisition regions 311, 312, and 313 correspond to the right temporal region, the occipital region, and the forehead, the vectors 501 to 503 indicate the front-rear direction, the body axis direction, and the left-right direction, respectively. Therefore, the directions of the three axes being calculated are different depending on the set positions of the point-group acquisition regions 311, 312, and 313. Thus, it should be noted, depending on the setting of the point-group acquisition regions, the calculated three axes may be associated with different orthogonal three axes of the image space coordinate system that is included in the medical image data in advance.

According to the first embodiment, the surface point group is acquired for each separated point-group acquisition region, and this eliminates the necessity of additional steps for the initial registration, thereby improving the operability and the convenience of the surface registration.

In other words, according to the first embodiment, the surface point group of the patient is acquired for each of the separated regions, enabling simultaneous acquisition of both the patient orientation data necessary for the initial registration and the patient surface data used for the detailed registration. Accordingly, cutting of the operation procedure for performing the surface registration can reduce a burden on the user and improve the operability.

### Second Embodiment

There will now be described the surgical navigation system of the second embodiment.

The surgical navigation system according to the second embodiment has the same configuration as the system according to the first embodiment, but differs from the first embodiment in that the surgical navigation system is further provided with a posture input unit for accepting an entry of the patient's posture from the operator.

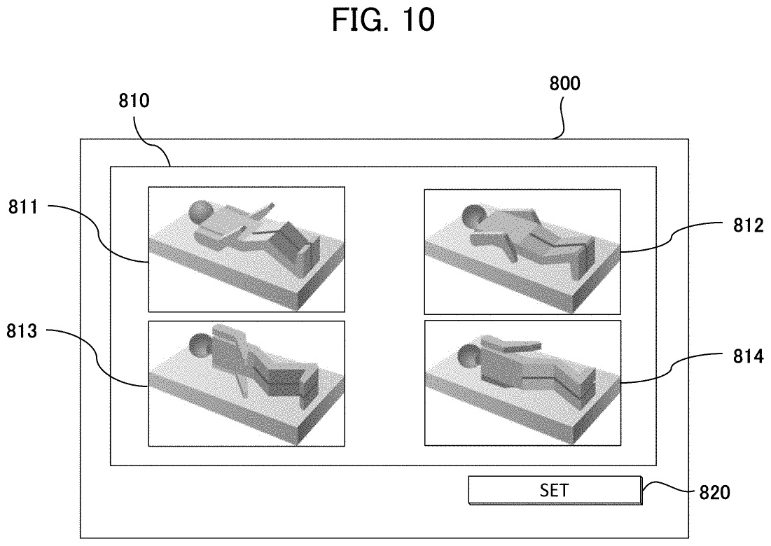

As shown in FIG. 10, the CPU 2 displays a patient posture entry screen 800 on the display device. The operator uses the mouse 8 to select a posture indicating the actual state of the patient 301, from the postures 811 to 814, and the CPU 2 accepts the entry via the mouse 8. Then, the CPU 2 implements functions of the posture input unit.

In accordance with the posture of the patient accepted by the posture input unit, the registration unit 21 selects three regions from two sets of regions facing each other (e.g., the forehead and the occipital region, and the right temporal region and the left temporal region), as the point-group acquisition regions 311, 312, and 313.

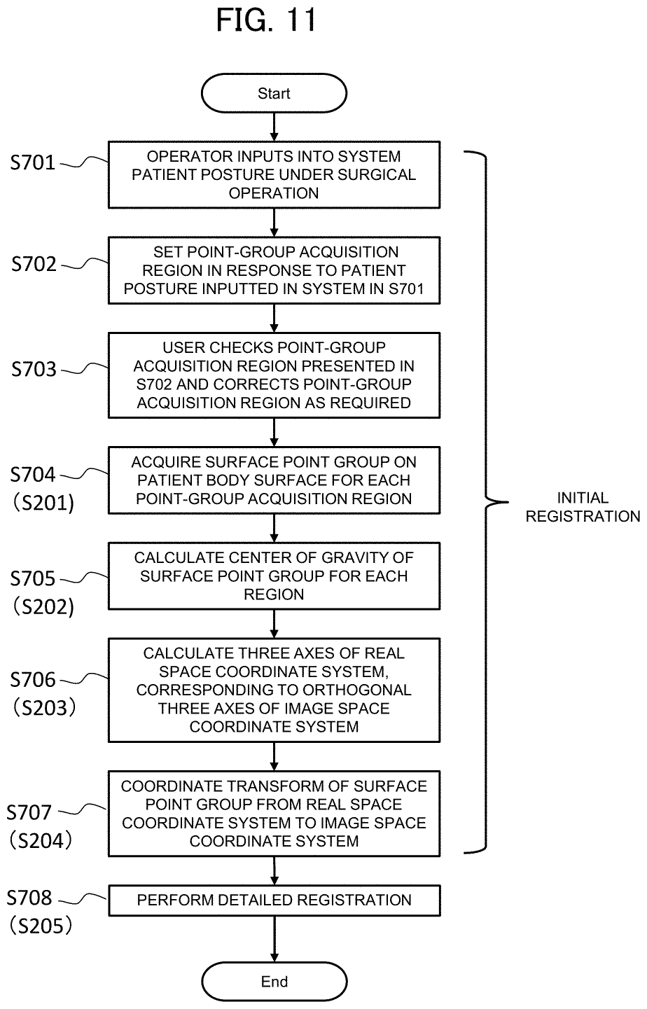

With reference to the flowchart of FIG. 11, there will now be described the processing of the posture input unit and the registration unit 21 according to the second embodiment.

### (Step S701)

First, the CPU 2 displays the patient posture entry screen 800 as shown in FIG. 10 on the display device. There are displayed a supine position 811, a prone position 812, a right lateral position 813, and a left lateral position 814, in the patient posture selection area 810 on the patient posture entry screen 800, and a set button 820 is further displayed. The operator selects on the patient posture entry screen 800, the posture corresponding to the patient posture under a surgical operation, by using the mouse 8, and then selects the set button 820, thereby entering the patient posture into the system.

### (Step S702)

In response to the patient posture entered in step S701, the system sets a predetermined appropriate point-group acquisition region, and displays the point-group acquisition region on the display device 6. For example, if the left lateral position is entered as the patient posture during the surgical operation, the system sets the forehead 311, the right temporal region 312, and the occipital region (not shown) as the point-group acquisition regions.

### (Step S703)

The operator checks the point-group acquisition regions presented by the system in step S702, and if there is any region where the surface point group is difficult to be acquired, the operator corrects the point-group acquisition region using the mouse 8 or a similar tool as required.

### (Steps S704 to S708)

Since the steps S704 to S708 are the same as S201 to S205 of the first embodiment, redundant descriptions will be omitted.

According to the second embodiment, the operator is only required to select the posture of the patient 301 to set an appropriate point-group acquisition region, thereby producing an effect that the procedure for setting the point-group acquisition regions can be simplified.

Configurations, operations, and effects of the surgical navigation system of the second embodiment, other than those described above, are the same as those of the first embodiment, and thus description thereof will be omitted.

What is claimed is:

1. A surgical navigation system comprising,
a storage unit configured to receive from an external device, a medical image being imaged for a patient, and to store the medical image,
a position detection sensor configured to detect position information of a point on a surface of the patient in real space, and
an registration unit configured to establish association between a position of the patient in real space and a position of a patient image in the medical image, wherein
the registration unit acquires from the position detection sensor, the position information of a plurality of points in three or more regions on the surface of the patient in real space, sets one representative position for each of the three or more regions, then using information of the representative position for each of the regions, performs initial registration to associate between an orientation of the patient in real space and an orientation of the patient image in the medical image, and thereafter, performs detailed registration to associate between the position of the patient in real space and the position of the patient image in the medical image, so that the surface shape of the patient represented by the positions of the plurality of points within the three or more regions matches the surface shape of the patient image in the medical image.

2. The surgical navigation system according to claim 1, wherein
the representative position in each of the regions, used for the initial registration, is calculated from the position information of the plurality of points for each of the regions.

3. The surgical navigation system according to claim 2, wherein
the representative position is a center of gravity of the plurality of points in the region.

4. The surgical navigation system according to claim 1, wherein
the three or more regions include three regions, out of two regions facing in the left-right direction of the patient and two regions facing in the front-rear direction of the patient, and the three regions are aligned along a circumferential direction of the patient.

5. The surgical navigation system according to claim 4, wherein
the registration unit performs the initial registration by selecting the facing regions out of the three or more regions, calculating a first vector connecting the representative positions respectively of the facing regions, calculating a second vector orthogonal to a plane including the representative positions of the three regions, also calculating a third vector orthogonal to the first and second vectors, and then the first, the second, and the third vectors, are respectively associated with the orthogonal three axes in image space of the medical image.

6. The surgical navigation system according to claim 1, wherein
the registration unit sequentially displays the three or more regions on a display device and prompts an operator to trace the surface of the patient in the regions with a pointer, and
the position detection sensor detects a position of the pointer with which the operator traces the surface of the patient, thereby detecting the position information of one or more points on the surface of the patient.

7. The surgical navigation system according to claim 6, wherein
the registration unit repeats an operation to determine the position being detected as the position information of a next point, when there is no already-acquired point within a predetermined distance from the position detected by the position detection sensor, until the position information of a predetermined number of points is acquired for the region.

8. The surgical navigation system according to claim 4, wherein
the registration unit selects in response to a posture of the patient, three regions out of the two regions facing in the left-right direction of the patient and the two regions facing in the front-rear direction of the patient, and acquires the position information of the plurality of points for the selected regions.

9. The surgical navigation system according to claim 4, further comprising a posture input unit configured to accept from an operator an input of a posture of the patient, wherein
the registration unit selects the three regions, in response to the posture of the patient accepted by the posture input unit.

10. A medical imaging system with a surgical navigation function comprising,
an imaging device configured to take an image of a medical image of a patient,
a storage unit configured to receive the medical image from the imaging device and to store the medical image,
a position detection sensor configured to detect position information of a point on a surface of the patient in real space, and
a registration unit configured to associate between a position of the patient in real space and a position of a patient image in the medical image, wherein
the registration unit acquires from the position detection sensor, the position information of a plurality of points in three or more regions on the surface of the patient in real space, sets one representative position for each of the three or more regions, then using information of the representative position for each of the regions, performs initial registration to associate between an orientation of the patient in real space and an orientation of the patient image in the medical image, and thereafter, performs detailed registration to associate between the position of the patient in real space and the position of the patient image in the medical image, so that the surface shape of the patient represented by the positions of the plurality of points within the three or more regions matches the surface shape of the patient image in the medical image.

11. A registration method of medical images for surgical navigation comprising,
receiving a medical image of a patient from an imaging device,
acquiring position information of a plurality of points within a region, as to three or more regions on a surface of a patient in real space,
setting one representative position for each of the three or more regions,
performing initial registration that associates between an orientation of the patient in real space and an orientation of a patient image in the medical image, by using information of the representative position for each of the regions, and
performing detailed registration that further associates between the position of the patient in real space and the position of the patient image in the medical image, so that a surface shape of the patient represented by the positions of the plurality of points in the three or more regions matches the surface shape of the patient image in the medical image.
